(19) United States
(12) Patent Application Publication
(10) Pub. No.: US 2019/0125257 A1
(43) Pub. Date: May 2, 2019

(54) SYSTEM AND METHOD FOR DETECTING AND MONITORING POST TRAUMATIC STRESS DISORDER (PTSD) USING MAGNETIC RESONANCE SPECTROSCOPY (MRS)

(71) Applicant: Translational Research Institute, Woolloongaabba (AU)

(72) Inventors: Carolyn E. Mountford, Robina (AU); Scott Quadrelli, Acacia Ridge (AU)

(73) Assignee: Translational Research Institute, Woolloongabba (AU)

(21) Appl. No.: 15/759,424

(22) PCT Filed: Sep. 12, 2016
(86) PCT No.: PCT/US2016/051220

§ 371 Date: Mar. 12, 2018

Related U.S. Application Data
(60) Provisional application No. 62/216,947, filed on Sep. 10, 2015.
(60) Provisional application No. 62/217,484, filed on Sep. 11, 2015.

(51) Int. Cl. A61B 5/00; A61B 5/055; G01R 33/465; G01R 33/46

(52) U.S. Cl. CPC A61B 5/4848; G01R 33/4625; G01R 33/465; A61B 5/055

(57) ABSTRACT

A system and method identifies Post Traumatic Stress Disorder by the use of magnetic resonance spectroscopy (MRS) to measure absolute and relative concentrations of metabolites in specific brain regions in the central nervous system or brain. The system and method can be used as a diagnostic tool for the assessment of PTSD. These chemical changes in the brain of those people suffering from PTSD are different from those suffering from head injury, chronic pain and other neurological conditions.

### CROSS-REFERENCE TO RELATED APPLICATION

This application claims the benefit of priority from U.S. Provisional Patent Application No. 62/217,484 filed Sep. 11, 2015. This application also claims the benefit of U.S. Provisional Application No. 62/216,947 filed Sep. 10, 2015. The contents of both applications are incorporated herein by reference.

### BACKGROUND OF THE INVENTION

The present invention relates to a system and method for detecting and monitoring PTSD using spectral data obtained using magnetic resonance spectroscopy.

Within this patent application are certain reference citations. These citations are incorporated herein by reference.

PTSD is an increasingly common disorder affecting especially military personnel. The traditional ways of detecting the disorder are by clinical metrics of post-concussion symptom scale, balance error scoring stem and cognitive assessment. Neuropsychological evaluation includes attention related skills, new learning and memory function and intellectual functioning. These detection methods are time consuming, involve some degree of subjectivity and thus possibly misdiagnosis, and are costly.

One somewhat quantitative method to diagnose PTSD is using a blood specimen which detects fatty-acid amide hydrolase (FAAH) activity which has been linked with arousability and aversive-memories extinction, which are believed to be two key features of PTSD. See Translational Psychiatry (2012) 2, e75; doi: 10. 1038/tp. 2012.1, published online 31 Jan. 2012.

### SUMMARY OF THE INVENTION

In accordance with the present invention, a system and method are provided for diagnosing PTSD using spectral data obtained using magnetic resonance spectroscopy. The method comprises acquiring spectral data of a region of the brain of a subject, comparing the acquired spectral data with reference spectral data from normal subjects, and determining whether selected molecules in the obtained spectral data are different relative to the reference spectral data, to indicate that the subject has PTSD.

The invention also provides a system for detecting PTSD, and for monitoring the subject and progress of treatment.

The selected molecules may be one or more of histidine, fucosylated glycans, lipids, γ-Aminobutyric acid (GABA), macro molecules (MM), glyceryphosphorylcholine (GPC), Phenylalanine (PE) and glutamate/glutamine.

The invention provides an effective and relatively objective in vivo method and system for detecting PTSD, noninvasively, in a relatively fast and robust manner which also does not require any communication, counseling or therapy with the subject, thereby reducing the added stress and trauma attendant to the traditional ways of detecting PTSD which remind subjects of the very disorder for which they are seeking treatment.

Other advantages will be apparent from the following description and drawings.

### BRIEF DESCRIPTION OF THE DRAWINGS

FIG. 1 is a three dimensional graph showing 2D COSY data from subjects known to have PTSD;

FIG. 2 is a table showing the elevated levels of certain molecules from subjects known to have PTSD; and

FIG. 3 is a three dimensional graph showing levels of fucose in healthy controls (upper) and subjects with PTSD, wherein the fucose I and III are diminished in the PTSD subjects relative to the controls.

### DESCRIPTION OF THE PREFERRED EMBODIMENTS

A preferred embodiment of the invention will be disclosed, but the invention will not be limited to this embodiment.

The present invention provides a method of detecting whether a subject has Post Traumatic Stress Disorder (PTSD) comprising the steps of: acquiring spectral data of the region of a brain of a subject; comparing the acquired spectral data with reference spectral data obtained from normal subjects who have not been diagnosed with PTSD; and determining whether selected molecules in the obtained spectral data differ in concentration relative to the reference spectral data to determine whether the subject has PTSD based on the comparison.

The selected molecules may comprise at least one of histidine, fucosylated glycans, lipids, γ-Aminobutyric acid (GABA), macro molecules (MM), glyceryphosphorylcholine (GPC), Phenylalanine (PE) and glutamate/glutamine. The acquired spectral data may be L-COSY 2D spectral data. The method may further comprise repeating the steps of acquiring, comparing and determining while the subject is undergoing treatment for PTSD, to determine the progress of treatment.

The invention also provides a system for detecting whether a subject has Post Traumatic Stress Disorder (PTSD) comprising: a spectrometer acquiring spectral data of the region of a brain of a subject; a comparator for comparing the acquired spectral data with reference spectral data obtained from normal subjects who have not been diagnosed with PTSD; and a processor for determining whether selected molecules in the obtained spectral data differ in concentration relative to the reference spectral data to determine whether the subject has PTSD based on the comparison.

The selected molecules may comprise at least one of histidine, fucosylated glycans, lipids, γ-Aminobutyric acid (GABA), macro molecules (MM), glyceryphosphorylcholine (GPC), Phenylalanine (PE) and glutamate/glutamine. The acquired spectral data may be COSY 2D spectral data. The system may comprise a memory for storing spectral data obtained from a subject from at least two different time periods, and wherein the processor compares two differently obtained spectral data to determine whether the subject is responding favorably to treatment.

Subjects who have been diagnosed with PTSD using conventional psychological methods can be used to develop a reference database by having their brains scanned with an MR spectrometer separate or as part of a clinical MM scanner of a magnetic resonance spectroscopy system. The system can be a 3Tesla (3T) or higher MRI scanner using a 32 or 64 or greater channel head coil. The pulse sequence used may be an L-COSY either 1D or 2D. Further details on how to acquire such data may be found in Ramadan S. et al., In Vivo Two Dimensional MR Spectroscopy Compares the Biochemistry of the Human Brain and Glioblastoma, and Radiology, 2011. 259(2): p. 540-9 and Mountford, C., et al. Six fucose-alpha(1-2) sugars and alpha-fucose assigned in the human brain using in vivo two-dimensional MRS. NMR Biomed, 2015. 28(3): p. 291-6.

The spectral data from normal subjects will comprise a reference database in which certain molecules will have certain reference concentrations. Subjects known to have PTSD by conventional diagnostic methods have been found to have different concentration of certain molecules in their brain compared to normal subjects.

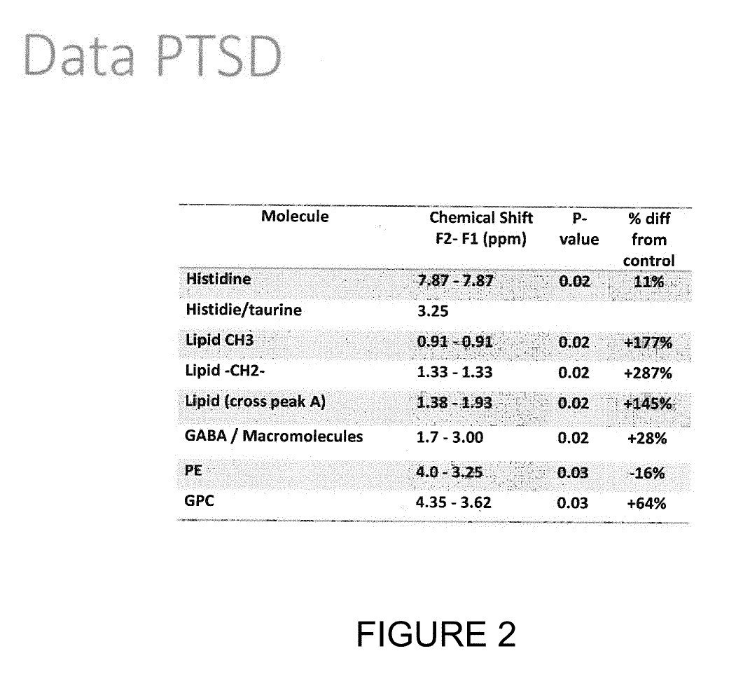

FIG. 2 comprises a Table of concentrations of certain molecules of PTSD subjects and how they differ from controls who are normal subjects. The last column of the Table shows the % difference of the molecule concentrations of the PTSD subjects relative to normal control subjects. The changes include but are not limited to those in Table 1 plus the fucosylated glycerns including Fuc1 and Fuc 4.

A subject to be evaluated for possible PTSD will undergo magnetic resonance spectroscopy of the brain to obtain the molecular concentration or ratio of the molecules listed and possibly other telltale marker molecules. By comparing the results with the reference molecule concentrations or ratios, a determination can be made on whether the suspected subject has PTSD in an efficient and robust manner. The comparison may also be made using a classifier method developed from a database.

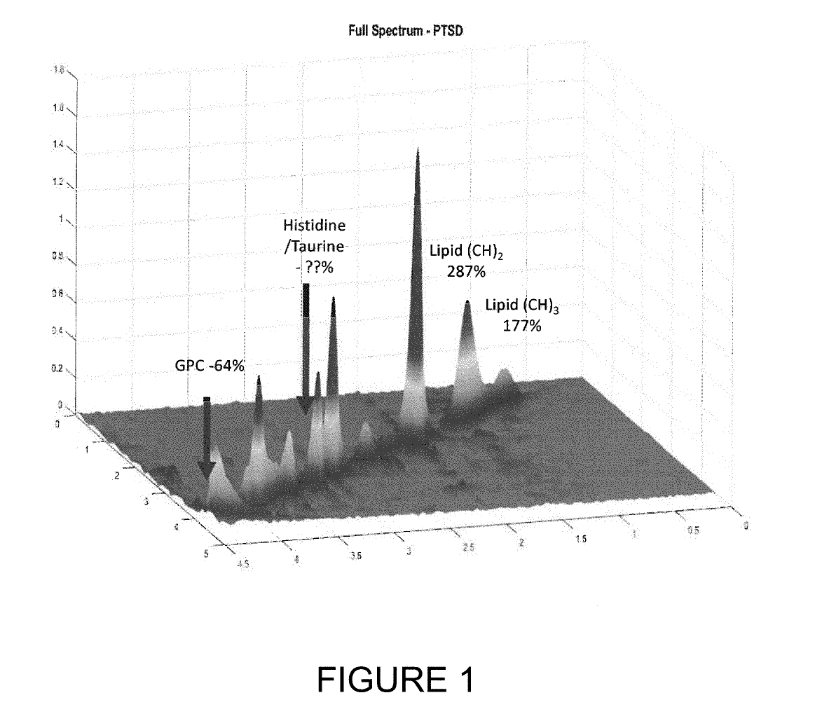

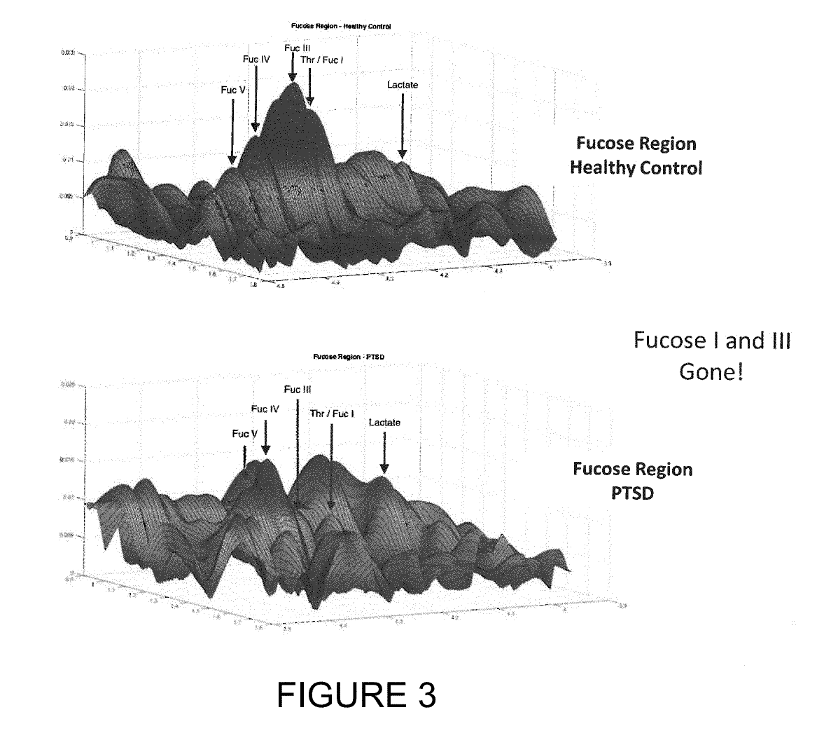

FIGS. 1 and 3 show three dimensional results of spectra from a subject diagnosed with PTSD, and showing different concentrations of certain molecules relative to normal controls.

The changes may differ depending on the region of the brain being examined. Classifiers may be developed for automated diagnosis if desired, instead of or in addition to manual diagnosis.

Diffuse axonal injury (DAI) from PTSD can arise from at least 10 different types of measurable deregulation or damage.

2D COSY provides specific DAI chemical changes providing a non-invasive and objective diagnosis more robust than conventional diagnosis methods.

The invention also includes treatment methods and monitoring to determine progress of subjects in treating the disorder. Personalized treatment approaches can be employed, as the diagnosis method reveals the precise chemical imbalances that need correction. For example, if the diagnosis result indicates inflammation, the condition can be treated with anti-inflammatory medications or other treatment, and periodically monitored by testing the subject to determine the rate of recovery.

Targeted intervention will provide early response with benefit in long-term neuropsychological and neuropsychiatric outcomes.

Although one embodiment has been described, the invention is not limited to this embodiment and variations may occur to those skilled in the art. The scope is limited only by way of the claims.

1. A method of detecting whether a subject has Post Traumatic Stress Disorder (PTSD) comprising the steps of:
acquiring spectral data of the region of a brain of a subject;
comparing the acquired spectral data reference spectral data obtained from normal subjects who have not been diagnosed with PTSD; and
determining whether selected molecules in the obtained spectral data differ in concentration relative to the reference spectral data to determine whether the subject has PTSD based on the comparison, wherein the selected molecules comprise at least one of histidine, fucosylated glycans, lipids, macro molecules (MM), glyceryphosphorylcholine (GPC), and Phenylalanine (PE).

2. (canceled) .

3. The method of claim 1, wherein the acquired spectral data is COSY 2D spectral data.

4. The method of claim 1, further comprising repeating the steps of acquiring, comparing and determining while the subject is undergoing treatment for PTSD, to determine the progress of treatment.

5. A system for detecting whether a subject has Post Traumatic Stress Disorder (PTSD) comprising:
a spectrometer acquiring spectral data of the region of a brain of a subject;
a comparator for comparing the acquired spectral data with reference spectral data obtained from normal subjects who have not been diagnosed with PTSD; and
a processor for determining whether selected molecules in the obtained spectral data differ in concentration relative to the reference spectral data to determine whether the subject has PTSD based on the comparison, wherein the selected molecules comprise at least one of histidine, fucosylated glycans, lipids, macro molecules (MM), glyceryphosphorylcholine (GPC), and Phenylalanine (PE).

6. (canceled)

7. The system of claim 5, wherein the acquired spectral data is COSY 2D spectral data.

8. The system of claim 5, further comprising a memory for storing spectral data obtained from a subject from at least two different time periods, and wherein the processor compares two differently obtained spectral data to determine whether the subject is responding favorably to treatment.
